(19) United States
(12) Patent Application Publication
(10) Pub. No.: US 2026/0097148 A1
(43) Pub. Date: Apr. 9, 2026

(54) HYDROPHOBICALLY-MODIFIED POLYMERS WITH BENZENEDIOL FUNCTIONAL GROUPS AND OXIDIZED FORMS THEREOF

(71) Applicant: Medcura, Inc., Riverdale, MD (US)

(72) Inventor: Matthew B. DOWLING, Baltimore, MD (US)

(21) Appl. No.: 19/114,883

(22) PCT Filed: Sep. 25, 2023
(86) PCT No.: PCT/US2023/075039

§ 371 Date: Mar. 25, 2025

Related U.S. Application Data
(60) Provisional application No. 63/377,089, filed on Sep. 26, 2022.

(51) Int. Cl. A61L 27/20; A61L 27/16; A61L 27/52; C08L 5/08

(52) U.S. Cl. CPC A61L 27/20; A61L 27/16; A61L 27/52; C08L 5/08; A61L 2400/04

(57) ABSTRACT

The present disclosure provides hydrophobically-modified biopolymers, including hydrophobically-modified chitosans, which comprise benzenediol groups, optionally where a portion are oxidized to the corresponding quinone. The biopolymers demonstrate a surprising ability to tune mucoadhesive properties, material cohesive properties, and hemostatic action of the material, to provide next generation hemostats, among other uses. Accordingly, in other aspects, the disclosure provides compositions comprising the modified biopolymers of the disclosure as well as methods for bleed and wound treatment.

### BACKGROUND

Biopolymer materials have been used for wound care and hemostasis, among other uses in health and personal care. Particularly for hemostasis (i.e., control of bleeding), the biopolymers must be engineered to balance several critical properties. For example, while chitosan shows good tissue adhesion properties, native chitosan itself exhibits minimal hemostatic action. Modified chitosans have been described with more advanced hemostatic properties, including hydrophobically-modified chitosans. See e.g., U.S. Pat. No. 8,932,560. However, hydrophobically-modified chitosans can lose properties of tissue adhesion and material cohesive properties, including when presented in flowable form. That is, while the modified chitosan can provide advanced hemostatic action, the ability of the modified chitosan to adhere to the wounded tissue and maintain cohesiveness especially in the presence of significant blood flow can be compromised.

Accordingly, modified biopolymers, such as modified chitosans, that balance tissue adhesion properties with hemostatic action and material cohesion are desirable for use in bleeding control, including for surgical bleeds. Other objectives of the present disclosure will be apparent from the following disclosure.

### SUMMARY OF THE INVENTION

The present disclosure provides hydrophobically-modified biopolymers comprising covalently attached hydrophobic grafts and benzenediol groups, and optionally oxidized forms thereof, along the polymer backbone. The hydrophobically-modified biopolymers include hydrophobically-modified chitosan (hm-chitosan), which comprise benzenediol groups optionally where a portion of the benzenediol groups are oxidized to the corresponding quinone. The biopolymers demonstrate a surprising ability to tune and balance the properties of mucoadhesion, material cohesion, and hemostatic action of the material, to provide next generation hemostats. Accordingly, in other aspects, the disclosure provides compositions comprising the modified biopolymers of the disclosure as well as methods and uses for bleed and wound treatment.

Accordingly, in one aspect, the present disclosure provides a hydrophobically-modified biopolymer comprising covalently attached hydrophobic grafts and benzenediol groups along the polymer backbone, optionally where a portion of the benzenediol groups are oxidized to the corresponding quinone.

In various embodiments, the hydrophobically-modified biopolymer is a modified-polysaccharide, such as chitosan, alginate, or cellulose. In some embodiments, the hydrophobically-modified biopolymer is hydrophobically-modified chitosan. Hydrophobic substituents that find use in accordance with this disclosure include (but are not limited to) saturated and unsaturated hydrocarbons. In some embodiments, the hydrophobic substituents are aliphatic, including straight chain or branched chain hydrocarbons. In various embodiments, the hydrophobically-modified biopolymer (e.g., chitosan) contains hydrophobic grafts (i.e., hydrophobic substituents) that are linear or branched hydrocarbons of from 6 to 18 carbon atoms. In exemplary embodiments, the hydrophobic grafts are linear hydrocarbons, and can be a uniform size or a combination of sizes.

In some embodiments, in the case of hm-chitosan, the hydrophobic grafts can be present on about 0.01% to about 15% or on about 0.01% to about 10% of the chitosan monomers. In some embodiments, the hm-chitosan has from about 0.1% to about 5%, or from about 0.5% to about 3% of chitosan monomers modified with a hydrocarbon chain independently selected from the range of C6 to C18. In some embodiments, the hm-chitosan comprises hydrophobic grafts selected from one or a combination of C8, C12, C14, C16, and C18. In some embodiments, the hydrophobic grafts are selected from C8, C10, and/or C12, and the grafts are present on about 0.1% to about 5%, or on about 1% to about 3%, or on about 1% to about 2% of the chitosan monomers. In some embodiments, the selection of hydrophobic grafts can provide another layer of tuning hemostatic action and material cohesion.

In various embodiments, the hydrophobically-modified biopolymer (e.g., hm-chitosan) can further comprise smaller hydrocarbon substituents (including C2, i.e., acetyl substituents as in chitin) to tune the density of positive charges as well as to tune the biodegradation rate of the material. In some embodiments, the smaller hydrocarbon substituents are selected from the range of a C1 to C4 hydrocarbons, which allows the chitosan to degrade more predictably from lysozyme activity in the body.

In accordance with this disclosure, the hydrophobically-modified biopolymer further comprises benzenediol groups substituted along the polymer backbone, and optionally where a portion of the benzenediol groups are oxidized to the corresponding quinone. For example, the benzenediol group can comprise catechol groups. The catechol moieties can be grafted to the biopolymer using hydrocaffeic acid or L-DOPA reagents, for example. The addition of benzenediol groups and oxidized forms thereof to the hydrophobically modified biopolymer increases the tissue adhesive properties (e.g., mucoadhesive properties). Further, such benzenediol groups added to the hydrophobically-modified biopolymer helps the biopolymers remain in solution and form gels that are easy to use.

In various embodiments, the benzenediol groups and oxidized forms thereof are grafted to the biopolymer at a density of from 0.1% to about 15% of polymer monomers. For example, the hydrophobically-modified biopolymer can be hm-chitosan and benzenediol groups and oxidized forms thereof are grafted to the hm-chitosan at a density of from about 0.1% to about 10% of chitosan monomers, or from about 0.1% to about 5% of chitosan monomers, or from about 0.1% to about 2% of chitosan monomers (e.g., about 0.8%). In various embodiments, about 10% to about 90% of the benzenediol groups are oxidized, or from about 25% to about 75% of the benzenediol groups are oxidized, or from about 30% to about 60% of the benzenediol groups are oxidized to the corresponding quinone. In various embodiments, the ratio of unoxidized benzenediol (e.g., catechol) to oxidized benzenediol (e.g. quinones) is about 1:2, or about 1:1; or in other embodiments, the ratio may be about 2:1, about 3:1, about 4:1 or about 5:1. As the level of oxidation increases, cohesiveness of the material also increases. Similarly, the greater the level of unoxidized molecules the greater the adhesiveness of the modified biopolymer.

The partial oxidation of the benzenediol groups allows for a large degree of tunability of both the adhesive and cohesive properties of the hydrophobically-modified biopolymer, e.g., in an aqueous hydrogel format.

In other aspects, the present disclosure provides compositions comprising the modified biopolymer of the instant disclosure. Generally, the compositions will further comprise a solvent or carrier, and in some embodiments are in the form of a hydrogel. In some embodiments, the solvent comprises water. Alternatively, the biopolymer composition may be formulated as a solid, powder, liquid, foam, or putty.

In various embodiments, the hydrophobically modified biopolymer is present in the composition at a concentration of from about 1 wt % to about 5 wt %, such as from about 1 wt % to about 3 wt %, based on the total weight of the composition. In various embodiments, the composition has a pH of from about 3.0 to about 6.0, or from about 3.5 to about 5.5, or from about 4.0 to about 5.5, or from about 4.5 to about 5.5. These pH levels provide for gels that are biocompatible, and further enables the modulation of the adhesive and cohesive properties of the gel by adjusting the pH within this range. In various embodiments, the solvent further comprises an organic or inorganic acid for adjusting the pH. The organic or inorganic acid in some embodiments may be selected from acetic acid, lactic acid, glycolic acid, glutamic acid, carbonic acid, citric acid, ascorbic acid, maleic acid, and combinations thereof.

In various embodiments, the composition further comprises one or more secondary polymers. In some embodiments, the secondary polymers are selected from gelatin, dextran, pectin, alginate, collagen, polyethylene oxide, gellan gum, polyvinyl alcohol, and combinations thereof. In some embodiments, the secondary polymer is in particle or granule form (e.g., gelatin granules). In embodiments, the composition may further comprise one or more additional components, such as an anti-flocculant agent, an anti-foaming agent, and an antimicrobial agent.

In other aspects, the disclosure provides a method for treating a bleed or wound, comprising applying the hydrophobically-modified biopolymer as described herein, or the composition described herein, to said bleed or wound. In various embodiments, the bleed or wound is a surgical bleed or may be a cavity bleed. In some embodiments, the bleed is at a location that is at risk of a pressure or compression related injury. Since the gel compositions disclosed herein do not swell following application, the compositions have desirable safety properties for application to sites at risk of pressure or compression related injury. In some embodiments, the surgical bleed is a scale 1 to 3 bleed (out of scale of 5). Due to the cohesive and adhesive properties of the material, and its hemostatic action, even moderate to severe bleeds can be controlled using the modified biopolymers and compositions of the disclosure.

In various embodiments, the composition can be applied in flowable form (e.g., hydrogel). Such materials are useful and easy to handle for treating surgical bleeds, including arterial bleeds and organ bleeds (e.g., liver bleeds). In some embodiments, the composition is a applied to a skin laceration. In other embodiments, the modified biopolymer or composition thereof is coated or incorporated with other materials such as bandages and wound dressings, especially for treating external wounds and bleeds.

Other aspects and embodiments of the instant disclosure will be apparent from the following detailed description.

### DESCRIPTION OF THE FIGURES

The above and other features, aspects, and advantages of the present invention are considered in more detail, in relation to the following description of embodiments thereof shown in the accompanying drawings, in which:

FIG. 1 compares hydrophobically modified chitosan with varied catechol substitutions. The vial on the right shows that a 2 wt % composition of a hydrophobically modified chitosan having 8% of chitosan monomer units comprising catechol grafts fails to make a stable gel. On the other hand, the vial on the left having 0.8% of monomer units comprising a catechol graft results in a stable gel.

FIG. 2 shows a UV spectral analysis of a composition of the present invention where two shoulders at 280 nm and 330 nm can be seen, which indicate the partial oxidation of the benzenediol groups. The peak at 280 corresponds to un-oxidized catechol. The peak at 330 corresponds to the quinone groups, which are the oxidized catechol groups.

FIG. 3 shows an example of a composition according to the disclosure (e.g., having partial oxidation of catechol groups) that is maintained at a pH of 5.4 and a composition that is titrated to pH of 12.0. The composition at pH of 5.4 provides strong hemostatic properties reflected in the full combination of the gel with the sample blood. On the other hand, the composition at pH 12.0 fails to interact with the blood. In the pH 12 gel, the catechol groups are fully oxidized and the combination of the high pH with the full catechol oxidation results in a structure that fails to react with the blood in the sample.

### DETAILED DESCRIPTION OF THE INVENTION

The present disclosure provides hydrophobically-modified biopolymers comprising covalently attached hydrophobic grafts and benzenediol groups, and optionally oxidized forms thereof, along the biopolymer backbone. The hydrophobically-modified biopolymers, including hydrophobically-modified chitosan (hm-chitosan), comprise benzenediol groups optionally where a portion of which are oxidized to the corresponding quinone. As described in detail below, the biopolymers demonstrate a surprising ability to tune or balance mucoadhesive properties, material cohesive properties, and hemostatic action of the material, to provide next generation hemostats. Accordingly, in other aspects, the disclosure provides compositions comprising the modified biopolymers of the disclosure as well as methods and uses for bleed and wound treatment.

Accordingly, in one aspect, the present disclosure provides a hydrophobically-modified biopolymer comprising covalently attached hydrophobic grafts and benzenediol groups along the biopolymer backbone, optionally where at least a portion of the benzenediol groups are oxidized to the corresponding quinone.

In various embodiments, the hydrophobically-modified biopolymer is a modified-polysaccharide, such as chitosan, alginate, or cellulose, all of which are abundant, natural biopolymers. The natural origin of these polysaccharides varies: cellulosics are found in plants, whereas chitosans and alginates are found in the exoskeleton or outer membrane of a variety of living organisms. Hydrophobically-modified biopolymers, including hydrophobically-modified chitosans and alginates, are described in U.S. Pat. Nos. 8,932,560; 8,668,899; and 10,179,145, which are each hereby incorporated by reference in its entirety. In some embodiments, the biopolymer is chitosan. Chitosan is a stable, robust, and durable biopolymer which is capable of retaining its functionality for extremely long storage periods at room temperature.

Chitosan is the common name of the linear, random copolymer that consists of β-(1-4)-linked D-glucosamine and N-acetyl-D-glucosamine. The molecular structure of chitosan is a linear backbone linked with glycosidic bonds. Chitosan can be derived by deacetylation of chitin, which may be obtained from one or more of crab, shrimp, krill, and crawfish. Commercial chitosan preparations are typically prepared by chemical de-N-acetylation of chitin under alkaline conditions. Depending on the source of the natural chitin (e.g., extracted from crustacean shells) and its production process, chitosan can differ in size (average molecular weight Mw) and degree of N-acetylation (% DA). While the poor solubility of chitosan in water and in common organic solvents restricts its applications, reactive amino groups in the chitosan backbone make it possible to chemically conjugate chitosan with various molecules and to modulate its properties for various applications.

The degree of deacetylation of chitin (to provide a native chitosan for modification) may generally range from about 40%-100%, or in some embodiments, from 50 to 100%, which determines the charge density and which makes the biopolymer readily reactive for modification. The charge density of chitosan is an important parameter for its tissue adherent properties. The amount of acetylation can be tuned by adding acetyl groups back to the chitosan, according to this disclosure. Thus, according to embodiments of this disclosure, the modified chitosan will have a free amine at about 40% of its monomers or more, or about 50% of its monomers of more, or at about 60% of its monomers or more. In various embodiments, the modified chitosan according to this disclosure comprises a free amine on about 40% to about 75% of it monomers, or from about 50% to about 75%, or from about 55% to about 75% of its monomers. The structure of chitosan (shown deacetylated) is depicted in Formula 1:

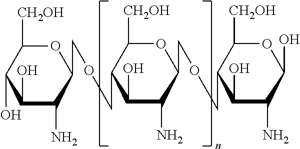

In various embodiments, the biopolymer is a hydrophobically modified chitosan (hm-chitosan). The chitosan can be a large molecular weight chitosan, or can be a medium molecular weight chitosan, or a small molecular weight chitosan. Generally, the molecular weight of the biopolymer (e.g., chitosan) will range from about 25,000 to about 1,500,000 grams per mole. In various embodiments, the molecular weight of the biopolymer (e.g., chitosan) ranges from about 40,000 to about 500,000 grams per more, or from about 50,000 to about 250,000 grams per mole, or from about 50,000 to about 100,000 grams per mole. As used herein, the term “molecular weight” means weight average molecular weight. Methods for determining average molecular weight of biopolymers include low angle laser light scattering (LLS) and Size Exclusion Chromatography (SEC).

In some embodiments, the biopolymer is chitosan having a low molecular weight of less than 150,000 Daltons (prior to modification). In other embodiments, the biopolymer is chitosan having a medium molecular weight of about 150,000 to about 350,000 Daltons (prior to modification). In yet other embodiments, the biopolymer is chitosan having a high molecular weight of about 400,000 Daltons or more (prior to modification).

The form of the natural biopolymers used may vary. For example, the hm-cellulosics may be formed from, without limitation, hydroxyethyl cellulose, hydroxypropyl cellulose, methyl cellulose, hydroxypropyl methyl cellulose, and/or hydroethyl methyl cellulose. Hm-chitosans may be prepared from, without limitation, the following chitosan salts: chitosan lactate, chitosan salicylate, chitosan pyrrolidone carboxylate, chitosan itaconate, chitosan niacinate, chitosan formate, chitosan acetate, chitosan gallate, chitosan glutamate, chitosan maleate, chitosan aspartate, chitosan glycolate and quaternary amine substituted chitosan and salts thereof. Hm-alginates may be prepared from, without limitation, sodium alginate, potassium alginate, magnesium alginate, calcium alginate, and/or aluminum alginate.

The hydrophobic modification of the chitosan backbone is through the association of an amphiphilic compound with the chitosan amino group, such that the hydrophobic tail of the amphiphilic compound is bound to the hydrophilic backbone structure. The processes by which hydrophobic modifications are added to the chitosan backbone have been previously described in U.S. Pat. Nos. 8,664,199; 8,668,899; 8,932,560; 9,066,885; 9,616,088; 10,179,145; 10,493,094; 11,274,194; 11,298,517, each of which is hereby incorporated by reference in its entirety.

Hydrophobic substituents that find use in accordance with this disclosure may be classified as saturated hydrocarbons or unsaturated hydrocarbons. In some embodiments, the hydrophobic substituents are aliphatic, including straight chain or branched chain hydrocarbons, or cyclic hydrocarbons. Generally, hydrophobic substituents are not substituted with hydrophilic groups (such as substituents comprising O, S, N, or halogen atoms). For example, according to this disclosure, benzenediol (e.g., catechol) and oxidized forms thereof are not considered hydrophobic substituents.

Hydrophobic substituents can be linear or branched hydrocarbons in various embodiments, and which may be saturated (e.g., joined entirely by single bonds, “alkanes”), or may comprise one, two, or three double bonds (“alkenes”).

In some embodiments, the hydrophobic substituents are linear or branched hydrocarbon chains. The hydrophobic modification of the chitosan backbone in some embodiments is through the association of a fatty aldehyde with the chitosan amino groups, such that the hydrophobic tail of the fatty aldehyde is bound with the hydrophilic backbone structure through an amine linkage. For example, chitosans can be reacted with alkyl aldehydes in aqueous acetic acid and ethanol, and the resulting Schiff bases can be reduced to stable secondary amines by addition of a reducing agent, such as sodium cyanoborohydride. See, U.S. Pat. No. 8,932,560, which is hereby incorporated by reference in its entirety. Alternatively, the hydrophobically-modified biopolymer is prepared using fatty acid anhydride chemistry, resulting in amide bonds with the chitosan biopolymer and the hydrocarbon chains. The amide bonds formed between chitosan and fatty acid anhydrides are shelf stable, even in the presence of dilute acids that are required to maintain solubility of the hydrophobically-modified chitosan. Accordingly, the modified biopolymers may be prepared using a one-pot synthesis, without the need for harsh reagents, including reducing agents. The materials can be precipitated following the reaction and dried for processing and incorporation into products, including solution, gels, and foams, among others. See U.S. Pat. No. 11,274,194, which is hereby incorporated by reference in its entirety.

In various embodiments, the hydrophobically-modified biopolymer (e.g., chitosan) contains hydrophobic grafts (i.e., hydrophobic substituents) that are linear or branched hydrocarbons of from 6 to 18 carbon atoms. In exemplary embodiments, the hydrophobic grafts are linear hydrocarbons, and can be a uniform size or a combination of sizes. In some embodiments, the hydrophobic grafts are present on about 0.01% to about 15% or about 0.01% to about 10% of the chitosan monomers. In some embodiments, the hm-chitosan has from about 0.1% to about 5%, or from about 0.5% to about 3% of chitosan monomers modified with a hydrocarbon chain independently selected from the range of C6 to C18. In exemplary embodiments, the hm-chitosan has from about 1% to about 3%, or from about 1% to about 2% of chitosan monomers modified with a hydrocarbon chain independently selected from the range of C6 to C18. In some embodiments, the hm-chitosan comprises hydrophobic grafts selected from one or a combination of C8, C12, C14, C16, and C18. In some embodiments, the hydrophobic grafts are selected from C8, C10, and/or C12, and the grafts are present on about 0.1% to about 5%, or on about 1% to about 3%, or on about 1% to about 2% of chitosan monomers.

In some embodiments, the selection of hydrophobic grafts can provide another layer of tuning hemostatic action and material cohesion. For example, C8, C10, and/or C12 acyl groups can be employed, which provide for effective hemostatic action, along with C14, C16 and/or C18 acyl groups that provide cohesive properties under exudate flow. The smaller hydrophobic grafts such as C8 are fluid at room and body temperature, allowing the biopolymer to effectively spread onto the tissue surfaces, whereas larger grafts such as C18 hold the chains together strongly even in the presence of high exudate or blood flow. See US 2020/0121825, which is hereby incorporated by reference in its entirety.

In various embodiments, the hydrophobically-modified biopolymer (e.g., hm-chitosan) can further comprise smaller hydrocarbon substituents (including C2, i.e., acetyl substituents as in chitin) to tune the density of positive charges as well as to time the biodegradation rate. In some embodiments, the smaller hydrocarbon substituents are selected from the range of a C1 to C4 hydrocarbon, which allows the chitosan to degrade more predictably from lysozyme activity in the body. This embodiments provide a material which can be left inside the body after treatment of the wound or bleed. See US 2020/0121825, which is hereby incorporated by reference in its entirety. In various embodiments, the density of the C1 to C4 hydrocarbon substituents (e.g., C2) along the biopolymer backbone (e.g., chitosan backbone) may be in the range of about 5% to about 50% of polymer monomers, or in some embodiments, in the range of about 20% to about 45% of polymer monomers. In some embodiments, the hydrophobically-modified polymer is hydrophobically-modified chitosan, and from about 5% to about 50% of the hm-chitosan monomers comprise an acetyl group, or from about 10% to about 40% of the hm-chitosan monomers comprise an acetyl group.

In accordance with this disclosure, the hydrophobically-modified biopolymer further comprises benzenediol groups substituted along the biopolymer backbone, and optionally where a portion a portion of the benzenediol groups are oxidized. For example, the benzenediol group can comprise catechol groups. The catechol moieties can be grafted to the biopolymer using hydrocaffeic acid or L-DOPA reagents, for example.

The addition of benzenediol groups to the hydrophobically-modified biopolymer compositions increases the tissue adhesive properties (e.g., mucoadhesive properties) of the hydrophobically-modified compositions. Further, such benzenediol groups added to the hydrophobically modified biopolymers help the biopolymer to remain in solution and form gels that are easy to use. The following is the chemical structure of native chitosan to which the benzenediol groups have been added by conjugation to available amines:

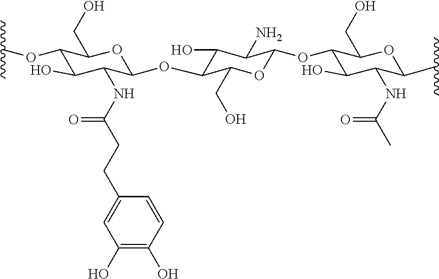 [SMILES: *OC1C(CO)OC(OC2C(CO)OC(OC3C(CO)OC(*)C(NC(C)=O)C3O)C(N)C2O)C(NC(=O)CCc2ccc(O)c(O)c2)C1O]

The following figure illustrates each monomer unit separated by brackets (m monomer comprises catechol moiety; n monomer comprises a free amine; and p monomer comprises acetyl:

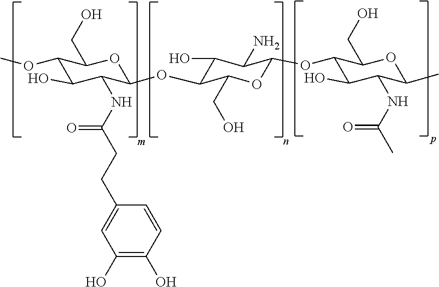

The following formula shows a chitosan molecule in which a benzenediol substituent has been fully oxidized to its corresponding quinone:

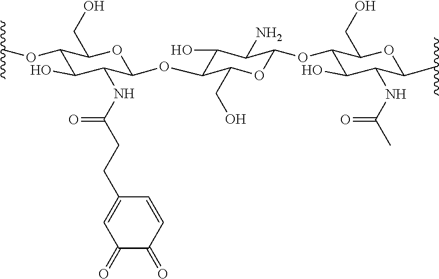 [SMILES: *OC1C(CO)OC(OC2C(CO)OC(OC3C(CO)OC(*)C(NC(C)=O)C3O)C(N)C2O)C(NC(=O)CCC2=CC(=O)C(=O)C=C2)C1O]

The following formula illustrates a hydrophobically modified chitosan of the disclosure, having benzenediol substituents, a portion of which are oxidized:

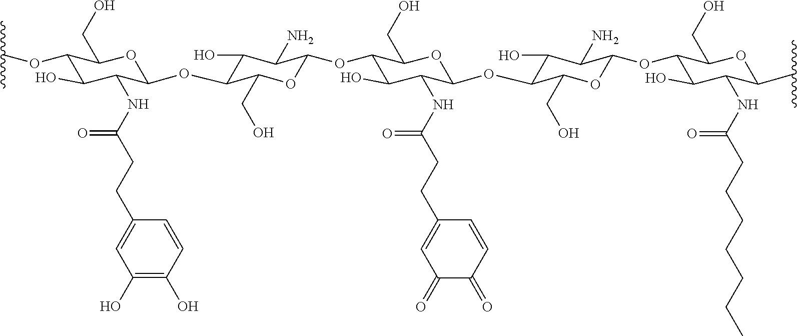 [SMILES: *OC1C(CO)OC(OC2C(CO)OC(OC3C(CO)OC(OC4C(CO)OC(OC5C(CO)OC(*)C(NC(=O)CCCCCCC)C5O)C(N)C4O)C(NC(=O)CCC4=CC(=O)C(=O)C=C4)C3O)C(N)C2O)C(NC(=O)CCc2ccc(O)c(O)c2)C1O]

According to this formula, hydrophobic grafts (C8 illustrated above) are present with benzenediol and its oxidized form in various ratios and densities, as described herein. The modified chitosan may also comprise C1-C4 groups (e.g., acetyl) as described elsewhere herein.

In various embodiments, the hydrophobically-modified chitosan can be described according to the following formula, in which an amount of chitosan monomers having a substituent comprising catechol are represented by an integer m; an amount of chitosan monomers having a substituent having a free amine are represented by an integer n; and an amount of chitosan monomers having a hydrophobic substituent are represented by an integer q:

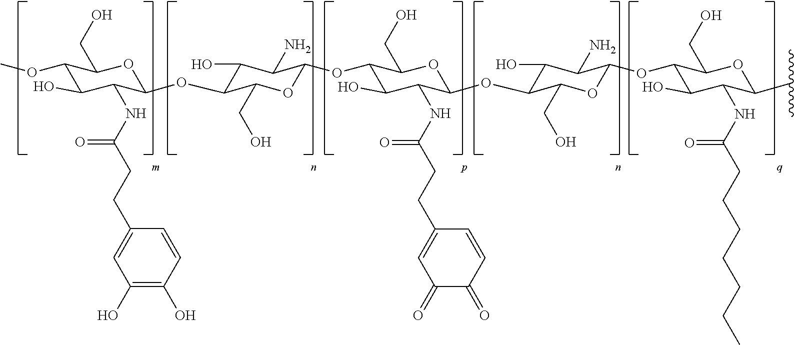

In various embodiments, the benzenediol groups and oxidized forms thereof (m and p) are grafted to the biopolymer at a density of from 0.1% to about 15% of biopolymer monomers. For example, the hydrophobically-modified biopolymer can be hm-chitosan and benzenediol groups and oxidized forms thereof (m and p above) are grafted to the hm-chitosan at a density of from about 0.1% to about 10% of chitosan monomers, or from about 0.1% to about 5% of chitosan monomers, or from about 0.1% to about 2% of chitosan monomers (e.g., about 0.8%). In various embodiments, about 10% to about 90% of the benzenediol groups are oxidized (monomer p in the formula above), or from about 25% to about 75% of the benzenediol groups are oxidized, or from about 30% to about 60% of the benzenediol groups are oxidized to the corresponding quinone. In various embodiments, the ratio of unoxidized benzenediol (e.g., catechol) to oxidized benzenediol (e.g. quinones) is about 1:2, or about 1:1 (e.g., a range of from about 1:2 to about 1:1); or in other embodiments, the ratio may be about 2:1, about 3:1, about 4:1, or about 5:1 (e.g., a range of about 1:2 to about 5:1, or a range of about 1:1 to about 5:1, or a range of about 2:1 to about 5:1). As the level of oxidation increases, cohesiveness of the material increase. Similarly, the greater the level of unoxidized molecules the greater adhesiveness of the modified biopolymer. The biopolymer in the foregoing formula may further comprise monomers having acetyl groups as already described.

The partial oxidation of the benzenediol groups allows for a large degree of tunability of both the adhesive and cohesive properties of the hydrophobically-modified biopolymer, e.g., in an aqueous hydrogel format. There are two measures of functionality of hemostatic gels: cohesiveness and adhesiveness. The cohesive nature of a hemostatic gel is exemplified by the ability of the gel to maintain integrity while under pressure. For example, in burst pressure tests in which a stream of water is directed at a gel, the higher cohesiveness of the gel results in the higher pressure necessary for the water to break through the gel. On the other hand, the adhesive nature of the gel can be measured in a similar way. The higher the level of adhesiveness, the more pressure that is required to dislodge the gel from a tissue or surface to which the gel is adhered. The most functional hemostatic gels have both higher cohesive values and higher adhesive values.

While hydrocarbon chains as hydrophobic grafts also provide a framework to tune or balance adhesive and cohesive properties of resulting hydrophobically modified biopolymer (as already described), partial oxidation of benzenediol groups along the backbone provide an additional layer of tunability, independent of the hydrophobic interactions conferred by the hydrophobic groups. For example, non-oxidized benzenediol groups amplify the adhesive properties of the biopolymer (e.g., mucoadhesive properties), whereas oxidized benzenediol groups amplify the cohesive properties of the biopolymer. In aggregate, the combination of hydrophobic modification, along with the addition of both non-oxidized and oxidized benzenediol groups onto the biopolymer backbone, results in a highly tunable hydrogel system for optimized adhesive and cohesive properties, as well as hemostatic action, so as to effectively treat bleeding from injured tissues (including moderate to severe surgical bleeds) and manage wound exudate. Cohesive properties of a gel can be measured based on the elastic modulus. For example, according to various embodiments, the elastic modulus of a hydrogel according to the disclosure ranges from about 50 to about 5,000 pascals. In some embodiments, the elastic modulus is less than about 4000 pascals, or less than about 2000 pascals, or less than about 1000 pascals, or less than about 500 pascals.

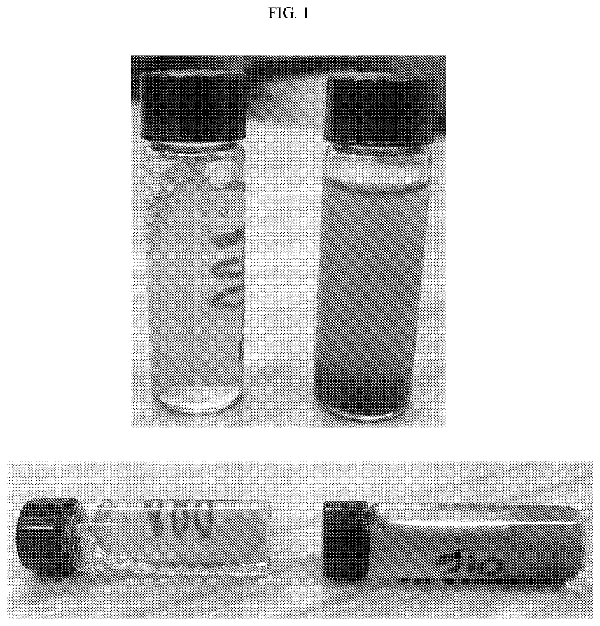

FIG. 1 shows a comparison between hydrophobically modified chitosan further comprising 8% catechol substitutions or 0.8% catechol substitutions (with respect to the number of monomers in the biopolymer). As shown in FIG. 1, the 8% catechol (right) fails to form a gel matrix and forms a precipitate. The 0.8% vial (left) shows a homogeneous and stable gel. While conjugation of catechol to chitosan has been described as exhibiting good tissue adhesive properties, it was surprising that these properties are exhibited at a very low level of catechol conjugation when added to hydrophobically-modified chitosan. As a result, in one embodiment, the hydrophobically modified biopolymer (e.g., chitosan) has benzenediol grafts (including oxidized forms thereof) in a concentration of at least about 0.1%, but less than about 10%, or less than about 8%, or less than about 5%, or less than about 3%, or less than about 2% of the monomer units. In various embodiments, the benzenediol grafts (including oxidized forms thereof) are present in the range of 0.2% to about 2% of polymer monomers, or in the range of about 0.5% to about 1.5%.

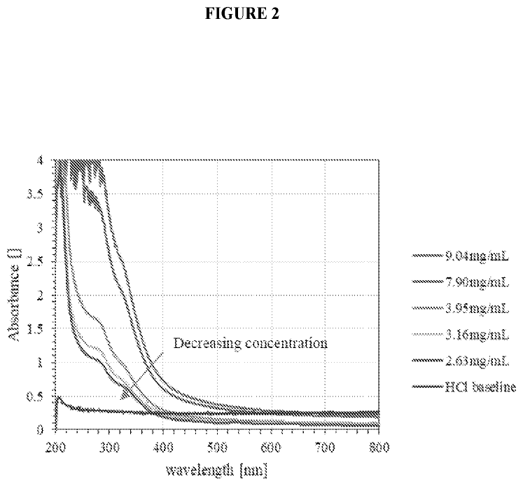

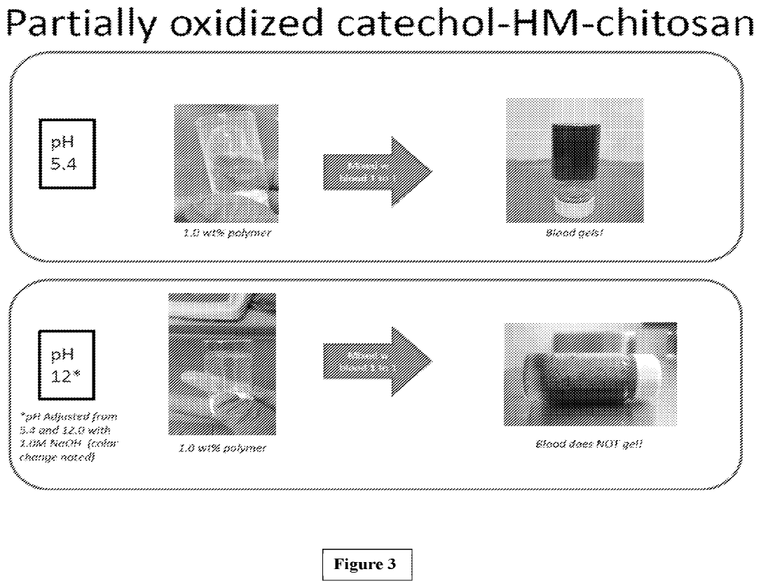

The properties of partial oxidation of catechol were unexpected and provide significant benefits to the material, specifically, it allows for modulation of adhesive and cohesive properties of HMC-C based on the level of oxidation of the benzenediol groups present in the molecule. The partially oxidized catechol groups on the backbone of the biopolymer are quinone functional groups (i.e., hydroxyls in the benzenediol are oxidized to quinone). The unoxidized hm-chitosan has no color, i.e, the gel is translucent. As oxidation increases, color of the gel ranges from clear to dark orange and has less fluid and hemostatic properties, as seen in FIG. 3. The level of oxidation can be monitored using UV spectral analysis, for example, as illustrated in FIG. 2.

An exemplary process for adding benzenediol groups to hydrophobically modified chitosan (HMC) involves the following steps. Hydrophobically modified chitosan is dissolved and/or suspended in distilled water. An acid, such as HCL, is added to the HMC to bring the solution to a desired pH and further solubilize the HMC. A catalyst such as 1-Ethyl-3-(3-dimethylaminopropyl)carbodiimide (EDC) hydrochloride and ethanol are added to the mixture. The source of the benzenediol, such as 3,4 dihydroxyhydrocinnamic acid (hydrocaffeic acid) (HCA) is added to the mixture, which is allowed to react for about one hour. While the reaction is taking place, an oxidizing agent such as NaOH is added to bring the pH to from about 7.0 to about 12.5 for several minutes. For example, the reaction can be allowed to proceed for up to 10 minutes, at which time the reaction is shocked with ethanol to stop oxidation. The higher the pH that the solution is allowed to achieve, the higher the level of oxidation. After the reaction is stopped, the resulting solution can be collected and processed, for example, dried and/or ground to a powder. Other processes for preparing materials (including powders) involving HMC containing benzenediol groups, a portion of which are oxidized, can be employed according to the present disclosure.

In other aspects, the present disclosure provides compositions comprising the hydrophobically-modified biopolymer of the instant disclosure (e.g., having benzenediol groups, a portion of which are oxidized, as described). Generally, the compositions will further comprise a solvent or carrier, and in some embodiments are in the form of a hydrogel. In some embodiments, the solvent comprises water. Alternatively, the biopolymer composition may be formulated as a solid, powder, liquid, foam, or putty.

For example, the biopolymer may be a solid, which may be lyophilized or may be a dehydrated solution or dehydrated foam or powder. Thus, the biopolymer may form a solid matrix. Foam formulations of hydrophobically modified chitosan which can be used are described in US 2021/0353501 or US 2022/0226625, which are hereby incorporated by reference in their entireties. In various embodiments, foams can be prepared or applied using propellants or CO₂ produced using a double barrel syringe system. In still other embodiments, the formulation is a putty as described, for example, in US 2014/0314706, which is hereby incorporated by reference in its entirety. Exemplary putty compositions can include biopolymers such as polyvinyl alcohol and ionic crosslinkers, such as sodium borate.

In various embodiments, the hydrophobically-modified biopolymer (for example, but without limitation, ground into a powder) is present in the composition at a concentration of from about 1 wt % to about 5 wt %, such as from about 1 wt % to about 3 wt %, based on the total weight of the composition.

In various embodiments, the composition has a pH of from about 3.0 to about 6.0, or from about 3.5 to about 5.5, or from about 4.0 to about 5.5, or from about 4.5 to about 5.5. These pH levels provide for gels that are biocompatible, and further allows one to modulate the adhesive and cohesive properties of the gel by adjusting the pH within this range. In various embodiments, the solvent further comprises an organic or inorganic acid for adjusting the pH. The organic or inorganic acid in some embodiments may be selected from acetic acid, lactic acid, glycolic acid, glutamic acid, carbonic acid, citric acid, ascorbic acid, maleic acid, and combinations thereof. In some embodiments, the acid comprises an organic acid selected from acetic acid, L-Lactic acid, and combinations thereof. For example, the solvent may comprise an organic acid present in a concentration of from about 0.03M to about 0.1M, or from about 0.04 to about 0.8M, and optionally about 0.05M.

In various embodiments, the composition further comprises one or more secondary polymers. In some embodiments, the secondary polymers are selected from gelatin, dextran, pectin, alginate, collagen, polyethylene oxide, gellan gum, polyvinyl alcohol, and combinations thereof. In some embodiments, the secondary polymer is in particle or “granule” form. In some embodiments, the composition comprises gelatin granules. In various embodiments, the particles or granules have an approximate mean diameter of from about 10 microns to about 2000 micron, or from about 50 to about 1000 microns, or from about 100 microns to about 750 microns, or from about 250 microns to about 750 microns. In some embodiments, the secondary polymer (e.g., gelatin granules) is present in a concentration of from about 0.01 wt % to about 5 wt %, or from about 0.05 wt % to about 2 wt %, or from about 0.1 wt % to about 1 wt % of the composition.

In embodiments, the composition may further comprise one or more additional components, such as an anti-flocculant agent, an anti-foaming agent, and an antimicrobial agent. For example, the composition can comprise EDTA as an anti-flocculant agent. The EDTA may be present in a concentration of from 1 ppm to about 1000 ppm, or from about 10 ppm to about 500 ppm, or from about 10 ppm to about 100 ppm. An exemplary anti-foaming agent is ethanol, which in some embodiments is present in a concentration of from about 0.1 wt % to about 5.0 wt %, or from about 0.1 wt % to about 2.0 wt %, or from about 0.1 wt % to about 1.0 wt % of the composition.

In an exemplary embodiment, the composition comprises hydrophobically-modified chitosan-catechol (HMC-C) (in embodiments described) in a concentration of from about 1.0 to about 4.0 wt %, and from about 0.2 to about 1.0 wt % of gelatin granules, and 0.01 to 0.10 M Acetic Acid, from about 30 to about 50 ppm EDTA; from about 0.1 to about 1.0 wt % Ethanol, and from about 85 wt % to about 98 wt % water.

In an exemplary embodiments, the composition comprises about 1.8 wt % HMC-C (with catechol grafted at a density of from about 0.5 to about 1.5% of chitosan monomers, to a medium or high molecular weight chitosan having about 35% C2, and about 1% C8, with respect to total chitosan monomers); about 0.5 wt % gelatin granules; about 0.05 M Acetic Acid; about 40 ppm EDTA; about 0.5 wt % Ethanol; and about 97% water. The example composition results in a gel as shown in on the left side of FIG. 1, where the gel composition on the left is homogenous and clear. In various embodiments the HMC-C is partially oxidized as described.

In other aspects, the disclosure provides a method for treating a bleed or wound, comprising applying the hydrophobically-modified biopolymer as described herein, or the composition described herein, to said bleed or wound. In various embodiments, the bleed or wound is a surgical bleed and may be a cavity bleed. For example, the bleed can be an arterial or venous bleed, or in some embodiments is an organ bleed. In some embodiments, the bleed is a liver bleed, or large or small bowel bleed. In some embodiments, the bleed is at a location that is at risk of a pressure or compression related injury. Since the gel compositions disclosed herein do not swell following application, the compositions have desirable safety properties for application to sites at risk of pressure or compression related injury. Such injuries may occur, for example, during surgeries selected from neurosurgery, orthopedic surgery, brain surgery, ocular surgery, otorhinolaryngological surgery, thoracic surgery, prostate surgery, thyroid surgery, cardiac surgery, vascular surgery, spine surgery, and gynecological surgery. For example, such injuries may occur in the context of spine surgeries, such as discectomy, laminectomy, laminotomy, lumbar decompression surgery, arthrodesis surgery, or anterior cervical discectomy. In various embodiments, such methods and uses of the composition avoid injury to one or more neurological structures, such as thecal sac compression, spinal cord compression, and nerve compression.

In embodiments, the composition is applied to a site selected from one or more of neurological structure, spinal cord, optic chiasm, spinal column, thecal sac, peritoneal sac, blood vessel, nerve, pulmonary artery, superior vena cava, inferior vena cava, brain tissue, bladder, cavernous nerve, muscle, bone, and joint. The surgery site may comprise bleeding selected from one or more of subcutaneous and muscular bleeding, bone bleeding, epidural bleeding, and large blood vessel bleeding. Other types of surgery for which the method can be used include prostatectomy, and the bleed may involves a cavernous nerve.

In some embodiments, the surgical bleed is a scale 1 to 3 bleed (out of scale of 5) In some embodiments, the bleed is at least a scale 3 bleed. In some embodiments, the bleed is a scale 4 bleed. Generally, the bleeding scale can be defined as: 0 (no bleeding, hemostasis): 1 (minimal bleeding); 2 (mild bleeding); 3 (moderate bleeding); 4 (severe bleeding), and 5 (extreme bleeding). In some embodiments, the patient has one or more factors that influence volume of blood loss during surgery selected from advanced age, higher body mass index, presence of osteoporotic bone, neuromuscular scoliosis, bone metastasis, and anti-coagulant therapy.

Due to the cohesive and adhesive properties of the material, and its hemostatic action, even moderate to severe bleeds can be controlled using the modified biopolymers and compositions of the disclosure.

In this context, bleeding scales (e.g., for skin lacerations) can be modeled as described in U.S. Pat. No. 10,283,015, which is hereby incorporated by reference. Devices can be used to simulate bleeds of different scales ex vivo by pumping natural or synthetic blood through a porous surface (e.g., determined by an interchangeable plate) and under different flow rates.

In various embodiments, the composition can be applied in flowable form (e.g., hydrogel). Such materials are useful and easy to handle for treating surgical bleeds. In other embodiments, the modified biopolymer or composition thereof is coated or incorporated with other materials such as bandages and wound dressings, especially for treating external wounds and bleeds.

As used herein, the term “about” means ±10% of an associated numerical value, unless the context indicates otherwise.

### EXAMPLES

The following Examples were conducted to investigate hydrophobically-modified chitosan materials with catechol functional groups and oxidized forms thereof.

### Example 1

In an exemplary process, a hydrophobically modified chitosan (HMC) having catechol functional groups was prepared essentially according to the following method. 10.0 g of HMC (C2/35, C8/1) was dissolved in distilled water. This HMC contains 35% of chitosan monomers having an amide (i.e., not deacetylated) and 1% of chitosan monomers have a C8 hydrocarbon graft. Thus, the HMC has available amines on about 64% of chitosan monomers. The chitosan had a molecular weight of about 400 kDa. Once dissolved, 18.4 Ml of 1M HCL was added, which increased viscosity and solubilized the HMC. In a separate container, 1.375 g EDC was dissolved in 500 mL of ethanol and 500 mL water. Once the EDC was dissolved, 0.650 g of hydrocaffeic acid was added to the EDC solution.

In one example, the EDC-hydrocaffeic acid solution was added to the HMC solution at a prescribed amount in order to prepare HMC-Catechol having about 8% of chitosan monomers having a catechol graft. In another example, the EDC-hydrocaffeic acid solution was added to the HMC solution at a prescribed amount, ten times lower than in the previous example, in order to prepare HMC-Catechol having about 0.8% of chitosan monomers having a catechol graft.

FIG. 1 compares the physical properties of an aqueous solution comprising 2 wt. % of the HMC-Catechol (8%) and an aqueous solution comprising 2 wt. % of HMC-Catechol (0.8%) (both at pH 4.5). The composition having 8% catechol fails to make a stable gel (right) due to lack of solubility. On the other hand, the vial on the left having 0.8% catechol results in a stable gel with intriguing mechanical properties.

Since catechol-modified chitosan is considered to have good mucoadhesive properties, and based on the results shown herewith HMC-Catechol, we investigated the ability to tune an HMC-Catechol to prepare a next generation hemostat that exhibits ideal properties of tissue adhesion, material cohesion, and hemostatic activity.

### Example 2

A hydrophobically modified chitosan (HMC) having catechol functional groups that are partially oxidized was prepared by dissolving 10.0 g of HMC (C2/35, C8/1) in distilled water. This HMC contains 35% of chitosan monomers having an amide (i.e., not deacetylated) and 1% of chitosan monomers have a C8 hydrocarbon graft. Thus, the HMC has available amines on about 64% of chitosan monomers. Once dissolved, 18.4 Ml of 1M HCL was added, which increased viscosity and solubilized the HMC. In a separate container, 1.375 g of EDC was dissolved in 500 mL of ethanol and 500 mL water. Once the EDC was dissolved, 0.650 g of hydrocaffeic acid was added to the EDC solution. The EDC-hydrocaffeic acid solution was added to the HMC solution. The resulting solution contained HMC-Catechol, with about 5 mol % catechol (i.e., about 5% of monomers have a catechol substituent). The HMC-Catechol was then partially oxidized by adding 40 ml of 2M NaOH to bring the reaction to a pH of 12.27 for about 10 minutes. In a comparative example, the reaction's pH was brought to 6.93 (Example 4). The composition where the pH is brought to 12.27 has more oxidized catechol groups than the one brought to 6.93. The reaction was stopped by shocking the solution with 1 L of ethanol.

The compositions were dried into powders. The powder was reconstituted to form a gel at a pH in the range of 4.5 to 5.5 (i.e., about 5.0). Adding catechol groups, a portion of which are oxidized, is believed to provide a balance between cohesive and mucoadhesive properties of the gel, while maintaining the hemostatic properties of the HMC. As oxidation increases, the cohesive properties of the gel increase. If oxidation proceeds to far, which we estimate to be beyond about pH 12.0 for more than 10 minutes before ethanol shock, the resulting powder no longer goes into solution.

### Example 3

A hydrophobically modified chitosan with partially oxidized catechol was prepared by dissolving 10.0 g of HMC (C2/35, C8/1) in distilled water. Once dissolved, 21.4 mL of 1M HCL was added, which increases viscosity and solubilizes the HMC. In a separate container, 1.376 g of EDC was dissolved in 330 mL of ethanol. Once the EDC was dissolved, 0.650 g of hydrocaffeic acid was added to the EDC solution. The EDC-hydrocaffeic acid solution was added to the HMC solution. The resulting solution contained HMC-Catechol. The HMC-Catechol was then partially oxidized by adding 8 mL of 2M NaOH to bring the reaction to a pH of 6.93. The reaction was stopped by shocking the solution with 1.5 L of ethanol.

Materials prepared substantially according to this example (prepared as hydrogels) were tested for tissue adhesive properties and material cohesive properties by testing burst pressure. Materials prepared substantially according to this example (prepared as hydrogels) were further tested for hemostatic action in vivo using a liver bleeding test. See, for example, U.S. Pat. No. 11,274,194, which is hereby incorporated by reference in its entirety. The resulting material is more adhesive material than HMC. It was determined that the materials show good properties with respect to tissue adhesion, material cohesion, and hemostatic action.

Analysis of an HMC-Catechol with partial oxidation (brought to pH 12.3) can be seen in FIG. 2, which is a UV spectral analysis where two shoulders at 280 nm and 330 nm can be seen and represent the partial oxidation state of catechol in the composition. The peak at 280 corresponds to un-oxidized catechol groups. The peak at 330 corresponds to the quinone groups, which are the oxidized catechol molecules.

### Example 4

Partially oxidized HMC-Catechol solution (essentially as prepared in Example 2) 1% (w/v) was prepared in 0.05M acetic acid in deionized water. The solution was viscous but flowable in nature. The pH of the solution was measured at 5.4. When mixed 50/50% (v/v) with citrated bovine whole blood and vortexed for 1 second, the resulting mixture formed a gel which holds its own weight upon vial inversion. See FIG. 3 (top). The same stock solution of partially oxidized HMC-Catechol solution, 1% (w/v), was titrated up to pH 12 by dropwise addition of 1.0 M NaOH. The solution (now containing more oxidized catechol) changed in color to a deep, dark orange, and became non-flowing in nature. This change in color from relatively clear, with a slightly red-to-orange hue, to deep, dark orange color is indicative of full oxidation of the catechol groups attached to the hydrophobically-modified chitosan backbone. When mixed 50/50% (v/v) with citrated bovine whole blood and vortexed for 1 second, the resulting mixture did not form a gel. The blood remained freely flowing and separated from the orange gel. The orange gel retained its bulk properties and largely did not interact with the blood, aside from a small layer of diffusion of blood into the surface of the gel. As shown in FIG. 3 (bottom), gels with fully oxidized catechol fail to interact with blood and create a gel complex. On the other hand, HMC-Catechol gels that are partially oxidized show good interaction with blood in forming gel complexes, while also displaying good tissue adhesion properties and material cohesive properties.

### Example 5

A pre-clinical pilot study was undertaken to investigate the safety and effectiveness of the HMC-Catechol gel following epidural application using an in-vivo ovine model. Ibis example uses an HMC-Catechol gel substantially as described in Example 1. The objectives of the study were to assess local histological response to the HMC-Catechol gel following lumbar laminectomy at 30-, 60- and 90-days post-operation and histopathological assessment of tissues from the spinal cord, exiting nerve roots and operative vertebral elements.

Animals underwent lumbar laminectomies at the L3 and L5 levels on Day 0 of study and operative sites received treatment with HMC-Catechol. In each animal, one site was treated ‘clinical case’ where excess HMC-Catechol not involved in the clot was removed per the instructions for use (IFU), and the second site was treated ‘worst case’ where no excess HMC-Catechol was removed. Animals were survived for 30-, 60-, or 90-days post-operation and subsequently euthanized. At necropsy, a postmortem laminectomy was performed at the intervening (non-operative, L4) level to allow for perfusion, and use of this level as a control. Tissues intended for histopathology and immunohistochemistry (IHC) analysis were collected, immersion fixed in 10% neutral buffered formalin (NBF).

The spinal tissues were trimmed, submitted for decalcification, processed and embedded in paraffin blocks. The resulting blocks were sectioned via microtome and mounted to glass slides. From each block, one slide was stained with hematoxylin and eosin (H&E), one slide was stained with Masson's Trichrome (MT), and one slide was immunohistochemically labeled for detection of Ionized Calcium Binding Adaptor Molecule 1 (IBA-1).

Microscopic evaluation of the lumbar spine with spinal cord from three (3) ovine animals treated with the HMC-Catechol, following laminectomy of the L3 and L5 levels and euthanized at 30, 60, or 90 days, under the conditions of this study, demonstrated the following salient findings. All tissue reaction observed at 30-, 60- and 90-days, at all operative laminectomy sites was similar and as expected for this surgical model and showed normal progression of healing across the timepoints. At the surgical access sites (all tissue dorsal to the epidural space), healing was characterized by fibrosis that tended to be replaced by bridging new bone, and minimal residual inflammatory cells, as well as minimal or mild amounts of residual HMC-Catechol associated with minimal numbers of macrophages, multinucleated giant cells and rare lymphocytes. The level of fibrosis was as expected in this surgical model and did not appear to be adversely affected by the HMC-catechol treatment.

Importantly, the amount of residual HMC-Catechol tended to decrease between each time interval, indicating progressive degradation. The pattern of absorption associated with the HMC-Catechol was relatively benign and associated with minimal or mild numbers of macrophages, multinucleated giant cells and rare lymphocytes. There was no difference in healing between “clinical” and “worst case” laminectomy sites. HMC-Catechol-related changes were limited to low grade lymphocyte, macrophage and multinucleated giant cell infiltrates with no effect on healing, suggesting excellent biocompatibility in the ovine model.

The tissue response at the epidural space was limited to fibrosis, minimal residual inflammation and occasional foci of residual HMC-Catechol. The fibrosis in the epidural space was scored as minimal to mild, with no evidence of excessive fibrosis in reaction to the HMC-Catechol. Epidural HMC-Catechol was detected at the 30- and 60-day time intervals but, importantly, there was no evidence of residual HMC-Catechol in the epidural space at 90 days.

Changes in the spinal cord consisted of nerve fiber degeneration and microglial reaction, both interpreted to be secondary to the surgical procedure and unrelated to the HMC-Catechol. Another important finding was that there was no evidence of ante-mortem compression-related injuries noted in the spinal cord. HMC-Catechol was not detected in the spinal cord or subdural spaces. Rare, inconsequential inflammatory cell infiltrates or adhesions were noted at the dura-leptomeninges in the subdural spaces. These changes were not associated with HMC-Catechol presence.

Treatment of the ovine lumbar spine with HMC-Catechol, after laminectomy and a survival period of 30-, 60- or 90-days under the conditions of this study showed excellent biocompatibility, advanced and nominal healing, and no adverse findings or safety concerns.

1. A hydrophobically-modified biopolymer, comprising a biopolymer comprising covalently attached hydrophobic grafts and benzenediol groups, and optionally oxidized forms thereof, along the biobiopolymer backbone.

2. The hydrophobically-modified biopolymer of claim 1, wherein the biopolymer is chitosan.

3. The hydrophobically-modified biopolymer of claim 2, wherein the chitosan is a medium or high molecular weight chitosan.

4. The hydrophobically-modified biopolymer of any one of claims 1 to 3, wherein the hydrophobic grafts comprise linear hydrocarbons of from 6 to 18 carbon atoms.

5. The hydrophobically-modified biopolymer of any one of claims 2 to 4, wherein the hydrophobically-modified biopolymer is a hydrophobically-modified chitosan (hm-chitosan) having from about 0.01% to about 10% of chitosan monomers modified with a hydrocarbon chain independently selected from a range of C6 to C18.

6. The hydrophobically-modified biopolymer of claim 5, wherein the hm-chitosan has from about 0.1% to about 5%, or from about 0.5% to about 3% of chitosan monomers modified with a hydrocarbon chain independently selected from the range of C6 to C18.

7. The hydrophobically-modified biopolymer of claim 5 or 6, wherein the hm-chitosan comprises hydrophobic grafts selected from one or more of C8, C12, C14, C16, and C18.

8. The hydrophobically-modified biopolymer of any one of claims 5 to 7, wherein from 5% to about 50% of the chitosan monomers comprise an acetyl group, and optionally from about 10% to about 40% of the hm-chitosan monomers comprise an acetyl group; optionally wherein the modified chitosan comprises a free amine on about 40% to about 75% of it monomers.

9. The hydrophobically-modified biopolymer of any one of claims 1 to 8, wherein the benzenediol groups comprise a catechol moiety and optionally oxidized forms thereof.

10. The hydrophobically-modified biopolymer of claim 9, wherein the catechol moiety is grafted to the hydrophobically-modified biopolymer using hydrocaffeic acid or L-DOPA reagents.

11. The hydrophobically-modified biopolymer of any one of claims 1 to 10, wherein the benzenediol groups and optionally oxidized forms thereof are grafted to the biopolymer at a density of from 0.1% to about 15% of biopolymer monomers.

12. The hydrophobically-modified biopolymer of claim 11, wherein the hydrophobically-modified biopolymer is hm-chitosan and benzenediol groups and optionally oxidized forms thereof are grafted to the hm-chitosan at a density of from about 0.1% to about 10% of chitosan monomers, or from about 0.1% to about 8% of chitosan monomers, or from about 0.1% to about 5% of chitosan monomers, or from about 0.1% to about 2% of chitosan monomers.

13. The hydrophobically-modified biopolymer of any one of claims 1 to 12, wherein at least some of the benzenediol groups are oxidized to a corresponding quinone.

14. The hydrophobically-modified biopolymer of claim 13, wherein from about 10% to about 90% of the benzenediol groups are oxidized, or from about 25% to about 75% of the benzenediol groups are oxidized, or from about 30% to about 60% of the benzenediol groups are oxidized to the corresponding quinone.

15. The hydrophobically-modified biopolymer of claim 14, wherein the hydrophobically-modified biopolymer is in powder form.

16. A composition comprising the hydrophobically-modified biopolymer of any one of claims 1 to 15 and a solvent.

17. The composition of claim 16, wherein the hydrophobically modified biopolymer is present in a concentration of from about 1 wt % to about 5 wt % based on the total weight of the composition.

18. The composition of claim 17, wherein the hydrophobically modified biopolymer is present in a concentration of from about 1 wt % to about 3 wt % based on the total weight of the composition.

19. The composition of any one of claims 16 to 18, wherein the solvent comprises water.

20. The composition of claim 19, wherein the composition has a pH of from about 3.0 to about 6.0, or from about 3.5 to about 5.5, or from about 4.0 to about 5.5, or from about 4.5 to about 5.5.

21. The composition of claim 20, wherein the composition is a hydrogel.

22. The composition of claim 20 or 21, wherein the solvent further comprises an organic or inorganic acid, which can be optionally selected from acetic acid, lactic acid, glycolic acid, glutamic acid, carbonic acid, citric acid, ascorbic acid, maleic acid, and combinations thereof.

23. The composition of claim 22, wherein the acid comprises an organic acid selected from acetic acid, L-Lactic acid, and combinations thereof.

24. The composition of claim 22 or 23, wherein the solvent comprises an organic acid present in a concentration of from about 0.03M to about 0.1M, or from about 0.04 to about 0.8M, and optionally about 0.05M.

25. The composition of any one of claims 16 to 24, further comprising one or more secondary polymers.

26. The composition of claim 25, wherein the secondary polymers are selected from gelatin, dextran, pectin, alginate, collagen, polyethylene oxide, gellan gum, and combinations thereof.

27. The composition of claim 26, wherein the secondary polymer is gelatin.

28. The composition of any one of claims 25 to 27, wherein the secondary polymer is in particle or granule form.

29. The composition of claim 28, wherein the particles or granules have a mean diameter of from about 10 microns to about 2000 micron, or from about 50 to about 1000 microns, or from about 100 microns to about 750 microns, or from about 250 microns to about 750 microns.

30. The composition of any one of claims 25 to 29, wherein the secondary polymer is present in a concentration of from about 0.01 wt % to about 5 wt %, or from about 0.05 wt % to about 2 wt %, or from about 0.1 wt % to about 1 wt % of the composition.

31. The composition of any one of claims 16 to 30, further comprising one or more of an anti-flocculant agent, an anti-foaming agent, and an antimicrobial agent.

32. The composition of claim 31, wherein the composition comprises EDTA as an anti-flocculant agent.

33. The composition of claim 32, wherein the EDTA is present in a concentration of from 1 ppm to about 1000 ppm, or from about 10 ppm to about 500 ppm, or from about 10 ppm to about 100 ppm.

34. The composition of any one of claims 31 to 33, wherein the anti-foaming agent is ethanol.

35. The composition of claim 34, wherein ethanol is present in a concentration of from about 0.1 wt % to about 5.0 wt %, or from about 0.1 wt % to about 2.0 wt %, or from about 0.1 wt % to about 1.0 wt % of the composition.

36. A method for treating a bleed or wound, comprising applying the hydrophobically-modified biopolymer of any one of claims 1 to 15, or the composition of any one of claims 16 to 35, to said bleed or wound.

37. The method of claim 36, wherein the bleed or wound is a surgical bleed.

38. The method of claim 37, wherein the surgical bleed is a cavity bleed or a bleed at a sit at risk of pressure or compression related injury.

39. The method of claim 38, wherein the surgical bleed is an injury due to spine surgery, neurosurgery, or vascular surgery.

40. The method of any one of claims 37 to 39, wherein the bleed is a scale 1 to 3 bleed.

41. The method of any one of claims 37 to 39, wherein the bleed is at least a scale 3 bleed.

42. The method of any one of claims 37 to 39, wherein the bleed is a scale 4 bleed.
